(19) United States
(12) Patent Application Publication
(10) Pub. No.: US 2022/0072313 A1
(43) Pub. Date: Mar. 10, 2022

(54) MODULAR BRAIN-COMPUTER INTERFACE

(71) Applicant: Braingrade Inc., Draper, UT (US)

(72) Inventors: Christian Hauptmann, Starnberg (DE); Adrian Weber, Hadamar-Steinbach (DE)

(21) Appl. No.: 17/467,310

(22) Filed: Sep. 6, 2021

(30) Foreign Application Priority Data
Sep. 10, 2020 (DE) 10 2020 211 366.1

(51) Int. Cl. A61N 1/36

(52) U.S. Cl. CPC A61N 1/36139; A61N 1/36067; A61N 1/36171; A61N 1/36082; A61N 1/36064

(57) ABSTRACT

A module for a brain-computer interface includes means for measuring neuronal activity in at least a portion of a population of neurons; means for stimulating at least the portion of the population of neurons; and means for local processing adapted to pre-analyze measured neuronal activity. A hub for a brain-computer interface includes means for powering a for the brain-computer interface by an alternating current (AC), wherein a charge transmitted in a cycle of the AC is below a tissue-damage threshold. A brain-computer interface comprises the module and the hub. A method for interfacing a brain and a computer includes measuring neuronal activity in at least a portion of a population of neurons; pre-analyzing, locally at a module for measuring and stimulating the portion of the population of neurons, measured neuronal activity; and receiving, at a hub for the brain-computer interface, a pre-analysis of the measured neuronal activity.

### CROSS REFERENCE TO RELATED APPLICATION

This application claims benefit to German Patent Application No. DE 10 2020 211 366.1, filed on Sep. 10, 2020, which is hereby incorporated by reference herein.

### FIELD

Described and disclosed herein are various systems, components, devices and methods for invasive neuronal stimulation as well as measurement of neuronal activity. The present invention provides technologies that enable therapies for treating neurological diseases like Parkinson's disease, dementia, psychiatric diseases and/or epilepsy (and/or others), but also therapies for treating movement impairments arising, e.g., after a stroke and/or a spinal cord injury. The present invention may also be used to support one or more brain functionalities.

### BACKGROUND

While current brain-computer interfaces bundle a variety of functionalities, they typically rely on several separate components and/or devices to provide one of these functionalities each. For example, a brain-computer interface as known in the state of the art may on the one hand comprise a data acquisition component and/or device that may be used to perform a measurement of neuronal activity in a population of neurons. On the other hand, the brain-computer interface may also comprise a separate, dedicated stimulation component and/or device that is used to provide stimulation, e.g., in the form of electric pulses, to the population of neurons. Further, such a brain-computer interface will additionally comprise a central processing component that centrally processes all data relating to the brain-computer interface (e.g., measurement as well as stimulation data). Examples for such components of current brain-computer interfaces providing a single functionality each are disclosed, e.g., in U.S. Pat. No. 9,566,439, US 2013/0289683, WO 2009/051965 and US 2018/0178010.

The current, fragmented design of brain-computer interfaces entails a number of drawbacks: Firstly, most current brain-computer interfaces are not implantable. The large number of components—which are often already bulky in and of themselves—alone simply forbids implementation, in effect restricting the use of current brain-computer interfaces to (quasi-)stationary scenarios. Secondly, current brain-computer interfaces are difficult to operate. For example, already the sheer number of different connections (e.g., in the form of different connectors, ports or sockets) required to connect the different components or devices to each other as well as to a central processing component presents a real challenge to any operator, provoking maloperation that could possibly lead to damage to the device or—even worse—harm to the patient or the operator. Thirdly, since central processing components as they are currently used in brain-computer interfaces need to process a wide variety of data (e.g., data regarding measured neuronal activity on the one hand and data regarding stimulation pulse patterns on the other), they can hardly adjust for and/or be tailored to the different functionalities provided by the other components and/or devices. Thereby, these central processing components fail to exploit the full potential of the different components and/or devices, such that most current brain-computer interfaces fall short of what is in principle technically possible.

### SUMMARY

In an embodiment, the present invention provides a module for a brain-computer interface includes means for measuring neuronal activity in at least a portion of a population of neurons; means for stimulating at least the portion of the population of neurons; and means for local processing adapted to pre-analyze measured neuronal activity. A hub for a brain-computer interface includes means for powering a for the brain-computer interface by an alternating current (AC), wherein a charge transmitted in a cycle of the AC is below a tissue-damage threshold. A brain-computer interface comprises the module and the hub. A method for interfacing a brain and a computer includes measuring neuronal activity in at least a portion of a population of neurons; pre-analyzing, locally at a module for measuring and stimulating the portion of the population of neurons, measured neuronal activity; and receiving, at a hub for the brain-computer interface, a pre-analysis of the measured neuronal activity. Another method for interfacing a brain and a computer includes powering at least one module for measuring and stimulating a portion of a population of neurons by an AC, wherein a charge transmitted in a cycle of the AC is below a tissue-damage threshold.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1: Exemplary brain-computer interface according to an embodiment of the present invention;

FIG. 2: Further exemplary brain-computer interface according to an embodiment of the present invention;

FIG. 3: Further exemplary brain-computer interface according to an embodiment of the present invention;

FIG. 4: Schematic representation of a coding and/or compression scheme according to an embodiment of the present invention; and

FIG. 5: Schematic representation of an application scenario involving one or more embodiments of the present invention.

### DETAILED DESCRIPTION

Embodiments of the present invention provide a brain-computer interface that at least partially ameliorates or even overcomes at least some of the drawbacks of known brain-computer interfaces.

In a first embodiment, the present invention relates to a module for a brain-computer interface.

Such a module comprises means for measuring neuronal activity in at least a portion of a population of neurons as well as means for stimulating at least the portion of the population of neurons. Hence, it may also be referred to as a measurement and stimulation module in the following. Additionally, the module comprises means for local processing adapted to pre-analyze measured neuronal activity.

Pre-analyzing measured neuronal activity may comprise categorizing at least one event comprised in the measured neuronal activity. Additionally or alternatively, pre-analyzing measured neuronal activity may comprise determining an onset time of at least one event comprised in the measured neuronal activity. In some embodiments, such pre-analyzing may suitably use spike detection.

Notably, a pre-analysis, e.g., a pre-analysis of measured neuronal activity (as it may be obtained by pre-analyzing measured neuronal activity as just described), does not necessarily render a full analysis moot (although it may in some embodiments). To the contrary, in some embodiments, a module according to the present invention may specifically comprise means for transmitting a pre-analysis of measured neuronal activity to a hub for the brain-computer interface. In particular, the means for local processing may then be adapted to coordinate with at least the hub for an analysis of the measured neuronal activity, such that the analysis is performed in a distributed manner.

In accordance with this concept of distributed analysis, the means for local processing may be located remote from at least one hub of the brain-computer interface. Preferably, the means for local processing may be located remote from all and any hubs of the brain-computer interface. For example, the means for local processing may be located in a same housing as the means for measuring and the means for stimulating. In some embodiments, the means for local processing may also be located in proximity to the respective population of neurons.

Such a module allows to consolidate several functionalities that would be distributed among several separate components and/or devices in current brain-computer interfaces. This ameliorates or even overcomes all of the drawbacks mentioned above: Firstly, a module according to the present invention may provide means for measuring neuronal activity in at least a portion of a population of neurons as well as means for stimulating at least the portion of the population of neurons in a single, small-scale device, making possible implantation and therefore non-stationary use of the brain-computer interface. Moreover, providing both measurement as well as stimulation functionalities in a single module may allow to dispense with separate connections (e.g., in the form of different connectors, ports and/or sockets) for the two functionalities, in turn making brain-computer interfaces employing modules according to the present invention more operator- and/or patient-friendly. In particular, the present invention thus significantly reduces the risk of maloperation and thereby also the risk of damage to the brain-computer interface as well as the risk of harm to the patient and/or operator. Finally, providing local processing means in modules for brain-computer interfaces in accordance with the present invention allows to specifically tailor these means for local processing to the functionalities that are to be provided by the module in which they are comprised (e.g., measurement and stimulation). Additionally, this may allow to relieve a central processing component and/or device of some of its tasks, thereby rendering any processing that is to be performed remote from the module quicker and more efficient. For example, thanks to means for local processing, modules according to the present invention may be able to deliver pre-analyses, perhaps in a standardized format, making it easier to process data from different modules. Overall, brain-computer interfaces employing modules according to the present invention may therefore function quicker and more efficient, but at the same time also more accurately.

In keeping with the idea of distributed analysis, the means for local processing may in some embodiments be adapted to pre-analyze measured neuronal activity using compression. This may render transmission of a pre-analysis (as it may be obtained by pre-analyzing measured neuronal activity), e.g., to a hub of a brain-computer interface more efficient as it may reduce the amount of data that needs to be transmitted.

In a similar vein, additionally or alternatively, a pre-analysis, e.g., a pre-analysis of measured neuronal activity (as it may be obtained by pre-analyzing measured neuronal activity as described above) may in some embodiments comprise at least one matrix. This may allow to exploit particularly efficient compression schemes, e.g., compression schemes at least partially based on video coding and/or compression schemes as will be described in more detail below.

The means for local processing may further be adapted to identify the module to a hub for the brain-computer interface. For example, the module may inform the hub of its functionalities and features, e.g., how many stimulation electrodes may be connected to it, the types and accuracy of measurements it may perform, maximum stimulation power, etc. This may allow to implement plug-and-play for the brain-computer interface as it is known, e.g., from modern computers to which any sort of peripheral may be connected, e.g., via USB or Bluetooth, without the need for any further intervention by the user. Rather, the computer and the plug-and-play device negotiate all parameters automatically, eventually providing the user with a ready-to-use system. Applying the concept of plug-and-play to brain-computer interfaces drastically improves their ease of use, in turn significantly reducing the risk of maloperation and hence damage to the interface and/or harm to the patient and/or operator.

In some embodiments, the means for local processing may additionally or alternatively also be adapted to control the means for stimulating. That is, just as the means for local processing are adapted to pre-analyze measured neuronal activity, they may also be adapted to perform and/or exert at least part of the control of the means for stimulating, such that stimulation—or the processing of stimulation commands and/or signals, respectively—is performed in a distributed manner. This may not only render stimulation—or the processing of stimulation commands and/or signals, respectively—quicker and more efficient. It also increases the modularity of the brain-computer interface because not all of the control needs to be performed and/or exerted by a central processing component and/or device.

Specifically, in some embodiments, the means for local processing may be adapted to control the means for stimulating based on stimulation signals received from a hub for the brain-computer interface. In some embodiments, the stimulation signals received from the hub may for example indicate one or more of a plurality of pre-defined stimulation pulse patterns to be applied using the means for stimulating. Similarly, the stimulation signals received from the hub may additionally or alternatively indicate at least one of an onset time, a duration, an intensity and/or a target portion of the population of neurons for one or more stimulation pulses to be applied using the means for stimulating. Using such stimulation signals that are then processed by the means for local processing in order to control the means for stimulating may allow to signal (e.g., transmit) stimulation signals in (possibly strongly) compressed form, rendering stimulation and the brain-computer interface as a whole more efficient. Moreover, the usage of such stimulation signals may also allow to signal (e.g., transmit) them in a standardized format. That is, e.g., a hub may not need to differentiate between stimulation signals it signals (e.g., transmits) to a lead and stimulation signals it signals (e.g., transmits) to another type of (measurement and) stimulation means such as a pad electrode.

In some embodiments, the stimulation signals may be coded and/or compressed. This may render transmission of the stimulation signals more efficient, increasing the achievable data throughput. This may be particularly advantageous as exchanging both pre-analyses as well as stimulation signals between one or more hubs and one or more modules may generate considerable traffic.

Preferably, the stimulation signals may be coded and/or compressed according to a video coding and/or compression scheme. For example, it may be straight-forward to apply such coding and/or compression schemes if the stimulation signals comprise at least one matrix. After all, a digital picture or frame of a video sequence is in effect a matrix. In some embodiments, every element (or entry) of such a matrix may represent a single stimulation contact. In some embodiments, all stimulation contacts represented by such a matrix may be part of the same lead, pad electrode and/or module according to the present invention. In other embodiments, stimulation contacts represented by such a matrix may be part of at least two different leads, pad electrodes and/or modules according to the present invention. Using existing coding and/or compression schemes as known, e.g., from video coding, may allow to code and/or compress signals such as stimulation signals particularly efficiently since these schemes are already well advanced. Additionally, using existing coding and/or compression schemes may avoid the need of developing a completely new coding and/or compression scheme, in turn rendering brain-computer interfaces employing modules according to the present invention cheaper and more economical.

According to the present invention, a module may further comprise means for connecting at least a second module for the brain-computer interface. That is, it may be possible to connect multiple modules in series rather than connecting each and every module to a hub directly. Considering that modules according to the present invention will typically be implanted, connecting modules to each other may allow to minimize the amount of cables that need to be routed through the body (wireless connections are often not viable within the body due to strong attenuation by the body). This may greatly benefit the safety of a brain-computer interface. Moreover, this may reduce the risk of damage and/or malfunction due to cable rupture or the like.

The means for connecting may be adapted to share (e.g., provide) power at least with the second module. Thereby, the need to provide every module with its own power supply may be circumvented. This is highly desirable as it allows to have, e.g., a single, central power supply that feeds all modules connected to, e.g., a given hub. This renders battery recharging and/or battery service particularly easy. Rather than recharging and servicing batteries of each module separately, the patient and/or operator may then only need to recharge and/or service a single battery. Considering that the modules according to the present invention will typically be implanted, this is particularly desirable since otherwise each and every module would need to be removed and reinserted into the body one by one, e.g., in a surgical procedure, to perform battery service for each of the modules separately. This may not only greatly increase the safety of brain-computer interfaces employing modules according to the present invention, but also the longevity of such brain-computer interfaces.

The means for connecting may comprise a female connector. This may benefit the safety of a brain-computer interface employing modules according to the present invention since—as opposed to male connectors—the risk of (internal) injuries by the module is decreased.

In some embodiments, the means for local processing may further be adapted to control the means for stimulating based at least in part on measured neuronal activity. That is, a closed-loop functionality may be implemented that allows to adapt stimulation based on measured neuronal activity. This may increase the effectiveness and precision of stimulation, providing for enhanced effects.

In a second embodiment, the present invention relates to a hub for a brain-computer interface. Such a hub comprises means for powering at least one module for the brain-computer interface by an alternating current, AC, wherein a charge transmitted in a cycle of the AC is below a tissue-damage threshold.

This allows to safely provide one or more modules, e.g., according to the first embodiment of the present invention, with power. On the one hand, a hub according to the second embodiment of the present invention allows to dispense with the need to provide every such module with its own power supply. As already explained above in the context of the (optional) capability of modules according to the first embodiment of the present invention to share power with other modules, this is desirable since, among other things, it avoids that modules need to be removed and reinserted again in a surgical procedure, e.g., for battery service. At the same time, as the hub provides power in the shape of an alternating current, wherein a charge transmitted in a cycle of the AC is below a tissue-damage threshold, the risk of serious damage to the patient implanted with modules and/or one or more hubs according to the present invention is minimized.

In some embodiments, a hub according to the second embodiment of the invention may further comprise means for identifying at least one module. As described above for a module according to the first embodiment of the present invention, this may allow to implement a plug-and-play functionality as it is known from modern computers.

Additionally or alternatively, the hub may comprise means for analyzing neuronal activity. As described above, the present invention may allow for analysis of measured neuronal activity in a distributed manner. That is, analysis may be performed between one or more modules according to the first embodiment of the invention and one or more hubs according to the second embodiment of the invention. To this end, a hub may further comprise means for receiving, from at least one module, a pre-analysis of neuronal activity measured by the at least one module. As already described above, such a pre-analysis of neuronal activity may comprise a category and/or an onset time of at least one event comprised in the neuronal activity measured by the at least one module.

A hub according to the present invention may also comprise means for connecting to an external control unit. Such an external control unit may be adapted to provide a user interface by means of which, e.g., an operator may control a brain-computer interface employing one or more hubs and one or more modules according to the present invention. For example, the external control unit may allow to view results of an analysis or pre-analysis of measured neuronal activity, or it may allow to adjust a stimulation pattern. It may also allow to define different categories for events that may be comprised in neuronal activity.

In some embodiments, a hub may further comprise means for coding and/or compressing a pre-analysis, such that the pre-analysis may be provided (e.g., transmitted) to an external control unit particularly efficiently. Preferably, such means for coding and/or compressing may operate according to a video coding and/or compression scheme, entailing the advantages detailed above.

In some embodiments, a hub according to the second embodiment of the present invention may comprise means for transmitting stimulation signals to the at least one module. The stimulation signals may indicate one or more of a plurality of pre-defined stimulation patterns to be applied by the at least one module. For example, the stimulation signals may indicate at least one of an onset time, a duration, an intensity and/or a target portion of the population of neurons for one or more stimulation pulses to be applied by the at least one module. As already described above, stimulation signals may be coded and/or compressed. To this end, a hub may then comprise means for coding and/or compressing the stimulation signals, preferably according to a video coding and/or compression scheme. Reference is made to the explanations above regarding the advantages such embodiments of a hub may provide.

In some embodiments, the stimulation signals may be based at least in part on a pre-analysis of neuronal brain-activity. That is, a closed-loop functionality may not only be implemented on the level of modules according to the present invention, but also at the level of hubs and/or external control units. Either way, a closed-loop functionality may allow to adapt stimulation based on measured neuronal activity. This may increase the effectiveness and precision of stimulation, providing for enhanced effects.

A hub for a brain-computer interface according to the second embodiment of the present invention may further comprise means for connecting at least a second hub for the brain-computer interface. Just as the present invention allows to connect modules according to its first embodiment in series, it may also allow to connect hubs according to its second embodiment in series. This may provide brain-computer interfaces employing hubs according to the present invention with a particularly high degree of modularity, in turn enhancing its usability and user-friendliness.

In some embodiments, the means for connecting may comprise a radio interface. A radio interface presents a particular convenient way of connecting two or more hubs. In particular, since a connection via a radio interface may be established ad hoc, i.e., e.g., without laying any cables, this further increases the modularity of brain-computer interfaces employing hubs according to the present invention, in turn also enhancing its usability and user-friendliness.

In another embodiment, the present invention relates to a brain-computer interface comprising at least one module according to the first embodiment of the invention and at least one hub according to the second embodiment of the invention.

Furthermore, in yet another embodiment, the present invention relates to a method for interfacing a brain and a computer. Such method comprises measuring neuronal activity in at least a portion of a population of neurons, pre-analyzing, locally at a module for the brain-computer interface adapted to measure and stimulate the portion of the population of neurons, measured neuronal activity and receiving, at a hub, a pre-analysis of the measured neuronal activity.

Finally, in another embodiment, the present invention relates to a method for interfacing a brain and a computer, the method comprising powering at least one module for measuring and stimulating a portion of a population of neurons by an alternating current, AC, wherein a charge transmitted in a cycle of the AC is below a tissue-damage threshold.

For the sake of brevity only a few embodiments will be described in the following. The skilled person will recognize that the specific features described with reference to these embodiments may be modified and combined differently and that individual features may also be omitted if they are not essential. The general explanations in the sections above will also be valid for the following more detailed explanations.

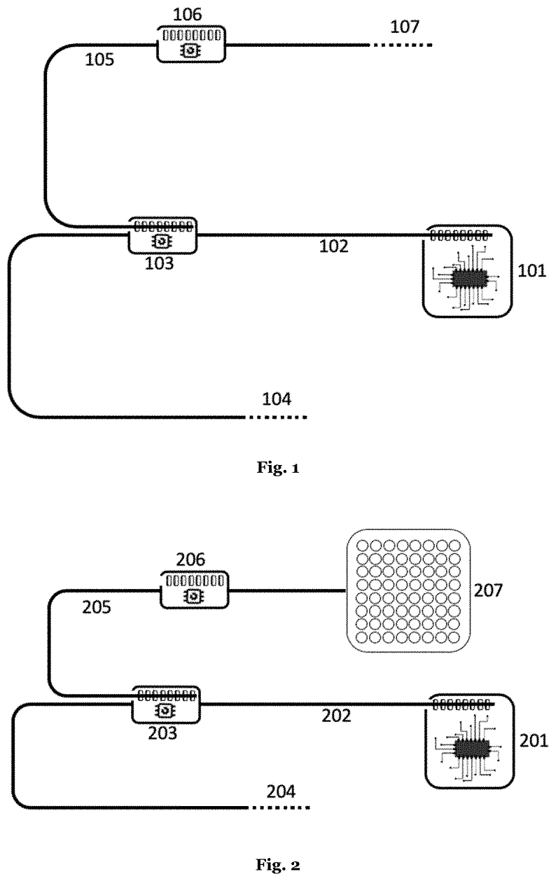

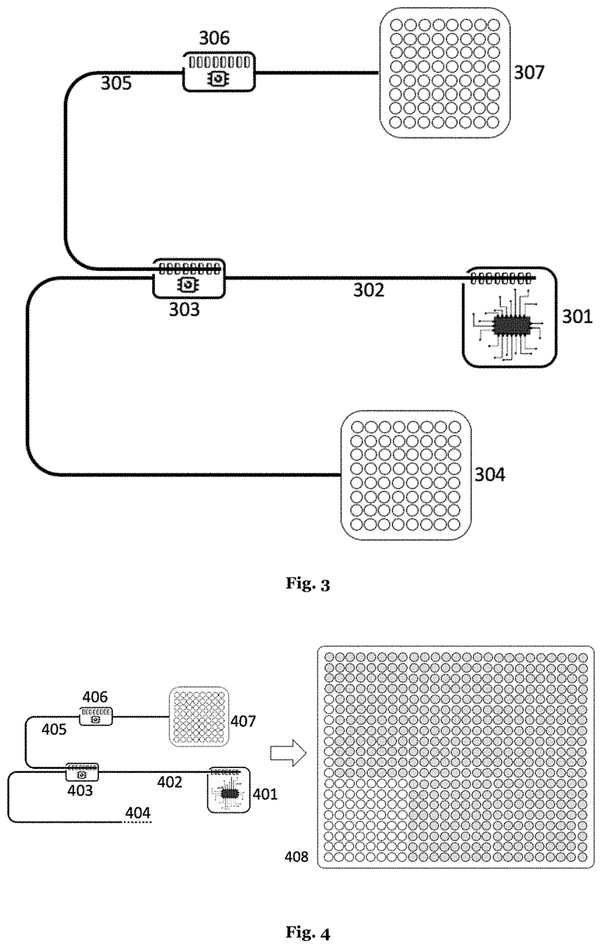

FIG. 1 shows an exemplary brain-computer interface according to an embodiment of the present invention. The brain-computer interface comprises a hub 101 that may, e.g., comprise a control unit and a power supply. The power supply could either be a built-in battery (rechargeable or not) or an external power supply (such as a mains adapter). Hub 101 is connected to leads 104, 107 that serve as modules according to the present invention. As shown in FIGS. 2 and 3 and discussed below, other configurations are possible, as well.

Hub 101 may be implanted. Hub 101 is connected to lead 104 by a connection cable 102. Specifically, connection cable 102 connects hub 101 to electronics 103 of lead 104. Further, electronics 103 comprise means for connecting. Lead 107 is connected to these means for connecting by a connection cable 105. In particular, cable 105 thus connects electronics 103 of lead 104 and electronics 106 of lead 107.

Electronics 103, 106 render leads 104, 107 smart. That is, at least partly thanks to electronics 103, 106, leads 104, 107 may perform various functions: Leads 104, 107 may measure neuronal activity in at least a portion of a population of neurons. Leads 104, 107 may at the same time also be used to stimulate at least said portion of the population of neurons. Thanks to electronics 103, 106, leads 104, 107 may locally process corresponding stimulation signals received from hub 101 which may indicate one or more of a plurality of pre-defined stimulation pulse patterns that are to be applied. Such stimulation signals received from hub 101 may for example indicate at least one of an onset time, a duration, an intensity and/or a target portion of the population of neurons for one or more stimulation pulses that are to be applied. Electronics 103, 106 may also aid leads 104, 107 in compressing and/or decompressing, coding and/or decoding signals such as stimulation signals received from hub 101 or pre-analyses transmitted to hub 101. Moreover, leads 104, 107 may locally pre-analyze measured neuronal activity thanks to electronics 103, 106. Any pre-analysis obtained this way may then be transmitted to hub 101 for further processing and/or analysis. As such, leads 104, 107 and/or their electronics, respectively, may coordinate with at least hub 101 for analysis of measured neuronal activity, such that the analysis is performed in a distributed manner.

Moreover, electronics 103, 106 of leads 104, 107, respectively, allow to implement a plug-and-play functionality as known from modern computers and peripherals. Via electronics 103, 106, leads 104, 107 may identify themselves to hub 101, indicating their functionalities and, e.g., a number and configuration of stimulation contacts leads 104, 107 comprise.

Using means for connecting such as those comprised in electronics 103 (and 106), several leads such as leads 104, 107 could be interconnected, e.g., in series, to form a network of leads and neuronal contact points. As shown in FIG. 1, this allows for example that only a single cable (here: cable 102) of a first lead (here: lead 104) is immediately connected to hub 101, while all other leads (here: lead 107) are connected to said first lead (here: lead 104) rather than to hub 101.

FIG. 2 shows another exemplary brain-computer interface according to an embodiment of the present invention. Overall, the brain-computer interface of FIG. 2 is similar to that of FIG. 1, as also indicated by like reference numerals. That is, the brain-computer interface of FIG. 2 comprises a hub 201 similar to hub 101 of FIG. 1 and a lead 204 comprising electronics 203 that is connected to hub 201 by connection cable 202, similar to lead 104 of FIG. 1 comprising electronics 103 that is connected to hub 101 of FIG. 1 by connection cable 102. However, instead of lead 107 (comprising electronics 106), the brain-computer interface of FIG. 2 comprises a pad electrode 207.

Pad electrodes such as pad electrode 207 comprise an arrangement (e.g., an array) of measurement and stimulation contacts arranged on a carrier surface. That is, as opposed to leads such as leads 104, 107, 204, pad electrodes such as pad electrode 207 do not comprise measurement and stimulation contacts that are essentially arranged (e.g., stacked) along a single direction and/or line, but pad electrodes such as pad electrode 207 rather comprise measurement and stimulation contacts that are essentially arranged along two directions and/or in a plane. In other words, while leads such as 104, 107, 204 may be considered essentially one-dimensional, pad electrodes such as pad electrode 207 may be considered essentially two-dimensional. Yet, measurement and stimulation contacts of a pad electrode such as pad electrode 207 may function essentially the same way as measurement and stimulation contacts of a lead such as lead 104, 107, 204. As such, just like leads such as leads 104, 107, 204, pad electrodes such as pad electrode 207 are adapted to measure neuronal activity in at least a portion of a population of neurons as well as to stimulate at least the portion of the population of neurons. In some embodiments pad electrodes such as pad electrode 207 may be implanted.

Moreover, similar to leads 104, 107, 204 comprising electronics 103, 106, 203, pad electrode 207 comprises electronics 206. As such, electrode 207 also serves as a module according to the present invention, just as leads 104, 107, 204. Just as electronics 103, 106 render leads 104, 107 smart as described above, electronics 206 also render pad electrode 207 smart. The above explanations regarding functions that leads 104, 107 may perform apply mutatis mutandis also to pad electrode 207.

FIG. 3 shows another exemplary brain-computer interface according to an embodiment of the present invention. Overall, the brain-computer interface of FIG. 3 is similar to those of FIGS. 1 and 2, as also indicated by like reference numerals. However, the brain-computer interface of FIG. 3 comprises two pad electrodes 304, 307 rather than one pad electrode 207 and one lead 204 as the brain-computer interface of FIG. 2 or two leads 104, 107 as the brain-computer interface of FIG. 1. Similar to pad electrode 207 of FIG. 2, pad electrodes 304, 307 comprise electronics 303, 306. Notably, as shown in FIG. 3, pad electrode 307 is not immediately connected to hub 301, but rather to means for connecting comprised in electronics 303 of pad electrode 304 (by means of connection cable 305). Pad electrode 304 is in turn immediately connected to hub 301 (by means of connection cable 302).

Leads 104, 107, 204 and pad electrodes 207, 304, 307 and their respective electronics 103, 106, 203, 206, 303, 306, are powered by hub 101, 201, 301, respectively. In particular, hubs 101, 201, 301 may provide power in the shape of a high-frequency AC, wherein the charge transmitted in each cycle of the AC is small enough not to bear any risk of tissue damage (passive safety), but strong enough to feed (e.g., power) leads 104, 107, 204 and pad electrodes 207, 304, 307 as well as their respective electronics 103, 106, 203, 206, 303, 306.

The following assumptions and calculations may be relied on to ascertain the (passive) safety of such high-frequency AC powering of modules: Assuming the resistance inside a human body to be approximately 1000 Ohm, a peak to peak voltage may be chosen to be 0.25 V and a carrier frequency may be chosen to be 175 kHz, carrying a 0.0005 mC charge per cycle. Then, assuming that a typical leakage area has a diameter of 50 mm and therefore a size of 1.96 10⁻⁵ cm², a charge density would be 25.7 mC/cm², which is in any case below the critical limit of 30 mC/cm² (cf. Coffey, R. J. 2009 Artificial Organs 33(3): 208-220).

In another embodiment, the carrier frequency may be chosen to be 2 MHz, and the peak to peak voltage may be chosen to be 3 V. Assuming the same size for the leakage area, the resulting charge density would be limited to 27.0 mC/cm², which in any case is also below the critical limit of 30 mC/cm².

Generally, safety may be further enhanced by further increasing the carrier frequency and/or by reducing the peak to peak voltage.

FIG. 4 shows a schematic representation of a coding and/or compression scheme according to an embodiment of the present invention. As described above, stimulation signals may be transmitted from a hub to one or more modules (e.g., leads or pad electrodes) in a coded and/or compressed fashion. In particular, the employed coding and/or compression scheme may be similar to one used for coding and/or compression in visual systems. Specifically, each stimulation contact (e.g., of a lead or a pad electrode) may be regarded as a (potential) measurement and stimulation site. A brain-computer interface according to an embodiment of the present invention may support a pre-defined number of (potential) measurement and stimulation sites. This pre-defined number of (potential) measurement and stimulation sites may be described by a matrix of dimension n×m. Matrix 408 shown in FIG. 4 is an example of such a matrix. Each lead such as lead 404 and/or each pad electrode such as pad electrode 407 may be described by a sub-matrix—e.g., of dimension 1×8 for lead 404 (correspondingly comprising 8 stimulation contacts) or of dimension 8×8 for pad electrode 407 (correspondingly comprising 64 stimulation contacts)—of matrix 408 of dimension n×m. Then, if all (potential) measurement and stimulation sites are represented within matrix 408 of dimension n×m as sub-matrixes of corresponding dimensions, matrix 408 of dimension n×m may be handled like a visual imaging and stimulation system (e.g., a monitor). In particular, this may allow utilizing existing coding strategies.

If not all n×m entries of a matrix such as matrix 408 are necessary to represent the currently active measurement and stimulation sites (i.e., e.g., those measurement and stimulation sites that are or will be used within a certain timeframe), any unused entries of the matrix may be left in a pre-defined initial state (e.g., zero, illustrated by a gray shading of the corresponding entries in matrix 408 of FIG. 4). All other entries of a matrix such as matrix 408 may, e.g., reflect a currently or recently measured signal at the corresponding measurement and stimulation site.

The use of such a matrix is particularly beneficial to perform local processing adapted to pre-analyze measured neuronal activity. For example, in embodiments in which pre-analyzing may use spike detection, a matrix such as matrix 408 of dimension n×m may indicate detected spikes in a binary manner. For example, each entry that corresponds to a measurement and stimulation site at which a spike was detected may be set to 1, while each entry that corresponds to a measurement and stimulation site at which no spike was detected may be set to 0. Alternatively, an entry of a matrix such as matrix 408 may indicate a power level of a spectrum measured at the corresponding measurement and stimulation site (e.g., the power level may be represented by entries between 0 and 100).

In order to obtain a high temporal resolution for measurement and/or stimulation, the carrier frequency may need to be chosen very high, e.g., above 100 kHz to allow a resolution in the order of 10 μs.

To reduce the amount of data to be signaled (e.g., transmitted), stimulation signals may be standardized, e.g., they may be represented by bi-phasic pulses with a certain width and amplitude, such that only the onset of a respective stimulation pulse may need to be encoded and signaled (e.g., transmitted). Then, a small number of channels (e.g., 2-4) suffices to control a large number of measurement and stimulation sites.

As mentioned before, a coding and/or compression scheme to be employed may be at least partially based on well-established coding and/or compression schemes as used in coding and/or compressing moving pictures (e.g., animated images or movies). For example, advanced coding and/or compression schemes like H.264, H.265 or AV1 may be used.

The concepts just described with a view to stimulation signals may be applied to measurement signals, as well, e.g., to pre-analyses that are to be transmitted, e.g., to a hub. For example, electronics such as 103, 106, 203, 206, 303, 306 of the embodiments of FIGS. 1-3 may be capable of identifying events in measured neuronal activity using spike detection or burst detection. These events may then be categorized. Only an onset time and a category of the respective event may be transmitted to the hub to reduce the amount of data to be transmitted. For example, local field potentials typically reflect important neuronal activity in a frequency band from 0-100 Hz. Hence, a carrier frequency of 100 kHz may be sufficient to transmit corresponding events to a hub. For a single channel, 200 Hz may be required to detect events at 100 Hz. Therefore, in order to transmit 32 channels with a bandwidth of 100 Hz each, a carrier frequency of 6,400 Hz would already be sufficient; a carrier frequency of 100 kHz would hence be more than enough. To further reduce the amount of data to be transmitted, coding and/or compression schemes (such as H.264, H.265 or AV1) may be applied, as described above.

Generally, hubs according to the present invention may provide power to and control modules according to the present invention. Since it may be possible to connect two or more modules in series as described above, a hub may only need to provide for a limited number of connections (e.g., in the form of different connectors, ports and/or sockets), e.g., 8 connections.

Two or more hubs, possibly including implanted hubs, may communicate with each other using proprietary or standard, possibly wireless, communication protocols, e.g., in case one hub is located in the brain of a patient and a second hub is located in a limb of the patient.

Hubs according to the invention may provide for a closed-loop functionality using suitable algorithms which may run on the hub. In some embodiments, known tools for pattern detection may be employed, in particular if measurement and stimulation sites are represented by a matrix such as matrix 408 of dimension n×m, such that techniques known, e.g., from image processing may be applied as described above.

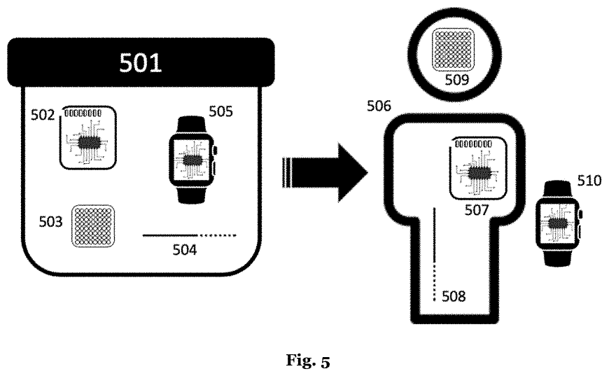

FIG. 5 shows a schematic representation of an application scenario involving one or more embodiments of the present invention. More particularly, FIG. 5 shows a toolbox 501. Toolbox 501 comprises one or more hubs such as hub 502, pad electrodes such as pad electrode 503, leads such as lead 504 and external control units such as external control unit 505. From a toolbox such as toolbox 501, an expert (e.g., a physician) may choose components that match the actual needs of a patient. Thus, toolbox 501 offers greatest flexibility and scalability for implementing smart neuronal measurement and stimulation in accordance with embodiments of the present invention.

Considering for example patient 506 shown in FIG. 5, a hub 507 may be implanted in a chest of patient 506. Hub 507 may be connected to (implanted) lead 508 (functioning as a module in the sense of the present invention) as well as to pad electrode 509 (also functioning as a module in the sense of the present invention). Furthermore, hub 507 is connected to external control unit 510. Measurement and stimulation modules 508, 509 are adapted to locally pre-analyze measured neuronal activity. As described above, performing such a pre-analysis at measurement and stimulation modules 508, 509 allows identifying events, such that, e.g., only an onset time and a category of the respective event may be transmitted to hub 507, thereby reducing the amount of data to be transmitted. Moreover, measurement and stimulation modules 508, 509 are adapted to process stimulation signals obtained (e.g., received) from hub 507. In some embodiments, such stimulation signals may also be informed by a pre-analysis of measured neuronal activity as performed, e.g., by modules 508, 509 (closed-loop functionality). External control unit 510 may provide further data, information and/or signals to hub 507 (e.g., regarding a heart rate and/or movement detection), e.g., via a wireless connection (e.g., via Bluetooth or Medical Implant Communication Service, MICS), via an optical connection (e.g., via optical signals that may be transmitted through the skin of a patient) and/or via encrypted communication channels. Conversely, external control unit 510 may also be used to read out data, information and/or signals (such as pre-analyses performed at and/or by modules such as modules 508, 509) from a hub such as hub 507 in order to perform an external analysis, e.g., regarding the performance of the system. An external analysis may thus be performed, e.g., at least partly based on one or more pre-analyses. For example, more computationally extensive analyses may be performed in order to compare obtained data with patterns in a memory, to check whether further actions are warranted (e.g. by trained personnel such as a physician or a nurse), or the like.

In some embodiments, an external control unit may be implemented in a smart watch, as is the case for external control units 505 and 510, that are accordingly well suited to provide additional data to a hub such as hub 502 or 507, e.g., regarding a heart rate and/or movement detection (e.g., using an accelerometer built into the smart watch). In other embodiments, an external control unit may be implemented in a mobile device, a personal computer (PC) or any other device.

Hubs such as hub 502 or 507 and modules such as modules 503, 504, 508, 509 may each come with dedicated firmware running specific algorithms, while external control units such as external control unit 505 or 510 may run a dedicated and user-friendly software, allowing, e.g., to configure hubs such as hub 502 or 507 using object orientated and intuitive programming.

For example, running such software, an external control unit such as external control unit 505 or 510 may be used to implement a closed-loop functionality at the level of a hub such as hub 502 or 507. In a similar vein, an external control unit such as external control unit 505 or 510 may also be used to define events triggering specific stimulation actions (i.e., e.g., pulses and/or patterns). Based on such software, an external control unit such as external control unit 505 or 510 may also be used to update the dedicated firmware of hubs such as hub 502 or 507 and/or modules such as modules 503, 504, 508, 509. In such embodiments, no external control units such as external control unit 505 or 510 may be necessary to normally operate the other components, though.

In some embodiments, the software running on an external control unit such as external control unit 505 or 510 may be adapted to first read out a configuration of any other, possibly implanted, components (e.g. a number of hubs, a number and type of connected modules, etc.). The software may also be adapted to ask for additional information (e.g., a goal of a configuration, location of modules, etc.) and may propose promising configurations from a library of configurations based thereon.

Additionally or alternatively, a hub such as hub 502 or 507 and/or an external control unit such as external control unit 505 or 510 may also be adapted to run a self-learning algorithm that uses measured neuronal activity and (output) stimulation signals, pulses and/or patterns to determine one or more actions that need to be taken to reach a pre-defined goal (e.g., movement of a specific limb, improved cognitive functionality). Using such a self-learning algorithm, a hub such as hub 502 or 507 and/or an external control unit such as external control unit 505 or 510 may learn how to most effectively reach the pre-defined goal.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A module for a brain-computer interface, comprising:
means for measuring neuronal activity in at least a portion of a population of neurons;
means for stimulating at least the portion of the population of neurons; and
means for local processing adapted to pre-analyze measured neuronal activity.

2. The module according to claim 1, wherein the means for local processing are adapted to pre-analyze measured neuronal activity by categorizing at least one event comprised in the measured neuronal activity and/or by determining an onset time of the at least one event comprised in the measured neuronal activity.

3. The module according to claim 1, wherein the means for local processing are adapted to pre-analyze measured neuronal activity using spike detection and/or compression.

4. The module according to claim 1, further comprising means for transmitting a pre-analysis of the measured neuronal activity to a hub for the brain-computer interface.

5. The module according to claim 4, wherein the means for local processing are adapted to coordinate with at least the hub for an analysis of the measured neuronal activity, such that the analysis is performed in a distributed manner.

6. The module according to claim 1, wherein the means for local processing are located in a same housing as the means for measuring and the means for stimulating.

7. The module according to claim 1, wherein the means for local processing are located in proximity to the population of neurons.

8. The module according to claim 1, wherein the means for local processing are further adapted to:
identify the module to a hub for the brain-computer interface; and/or
control the means for stimulating.

9. The module according to claim 1, wherein the means for local processing are adapted to control the means for stimulating based on stimulation signals received from a hub for the brain-computer interface.

10. The module according to claim 9, wherein the stimulation signals received from the hub indicate one or more of a plurality of pre-defined stimulation pulse patterns to be applied using the means for stimulating.

11. The module according to claim 9, wherein the stimulation signals received from the hub indicate at least one of an onset time, a duration, an intensity and/or a target portion of the population of neurons for one or more stimulation pulses to be applied using the means for stimulating.

12. The module according to claim 9, wherein the stimulation signals are coded and/or compressed, preferably according to a video coding and/or compression scheme.

13. The module according to claim 1, further comprising means for connecting at least a second module for the brain-computer interface.

14. The module according to claim 13, wherein the means for connecting are adapted to share power at least with the second module.

15. The module according to claim 13, wherein the means for connecting comprise a female connector.

16. The module according to claim 1, wherein the means for local processing are further adapted to control the means for stimulating based at least in part on measured neuronal activity.

17. A hub for a brain-computer interface, comprising:
means for powering at least one module for the brain-computer interface by an alternating current (AC),
wherein a charge transmitted in a cycle of the AC is below a tissue-damage threshold.

18. The hub according to claim 17, further comprising:
means for identifying the at least one module;
means for analyzing neuronal activity; and/or
means for connecting to an external control unit.

19. The hub according to claim 17, further comprising means for receiving, from the at least one module, a pre-analysis of neuronal activity measured by the at least one module, wherein the pre-analysis of neuronal activity preferably comprises at least one matrix.

20. The hub according to claim 19, wherein the pre-analysis of neuronal activity comprises a category and/or an onset time of at least one event comprised in the neuronal activity measured by the at least one module.

21. The hub according to claim 19, further comprising means for coding and/or compressing the pre-analysis.

22. The hub according to claim 17, further comprising means for transmitting stimulation signals to the at least one module.

23. The hub according to claim 22, wherein the stimulation signals indicate one or more of a plurality of pre-defined stimulation patterns to be applied by the at least one module.

24. The hub according to claim 22, wherein the stimulation signals indicate at least one of an onset time, a duration, an intensity and/or a target portion of the population of neurons for one or more stimulation pulses to be applied by the at least one module.

25. The hub according to claim 22, further comprising means for coding and/or compressing the stimulation signals.

26. The hub according to claim 22, wherein the stimulation signals are based at least in part on a pre-analysis of neuronal brain-activity.

27. The hub according to claim 17, further comprising means for connecting at least a second hub for the brain-computer interface.

28. The hub according to claim 27, wherein the means for connecting comprise a radio interface.

29. A brain-computer interface comprising at least one of the module according to claim 1 and at least one hub comprising means for powering the at least one module by an alternating current (AC), wherein a charge transmitted in a cycle of the AC is below a tissue-damage threshold.

30. A method for interfacing a brain and a computer, comprising:
measuring neuronal activity in at least a portion of a population of neurons;
pre-analyzing, locally at a module for measuring and stimulating the portion of the population of neurons, measured neuronal activity; and
receiving, at a hub for the brain-computer interface, a pre-analysis of the measured neuronal activity.

31. A method for interfacing a brain and a computer, comprising:
powering at least one module for measuring and stimulating a portion of a population of neurons by an alternating current (AC),
wherein a charge transmitted in a cycle of the AC is below a tissue-damage threshold.
